(19) United States
(12) Patent Application Publication
(10) Pub. No.: US 2010/0016614 A1
(43) Pub. Date: Jan. 21, 2010

(54) PROCESS FOR THE PREPARATION OF PERINDOPRIL ERBUMINE

(75) Inventors: Zoran Ham, Trbovlje (SI); Borut Furlan, Ljubljana (SI)

Correspondence Address: ARENT FOX LLP, 1050 CONNECTICUT AVENUE, N.W., SUITE 400, WASHINGTON, DC, 20036, US

(73) Assignee: LEK PHARMACEUTICALS D.D, LJUBLJANA (SI)

(21) Appl. No.: 12/063,493

(22) PCT Filed: Aug. 10, 2006
(86) PCT No.: PCT/EP2006/007926

§ 371 Date: Feb. 11, 2008

(30) Foreign Application Priority Data
Aug. 12, 2005 (SI) P200500232

(51) Int. Cl. C07D 209/12

(52) U.S. Cl. 548/452

(57) ABSTRACT

The present invention relates to a new process for the preparation of pure perindopril erbumine. The present invention also relates to a new process for the preparation of crystalline form D of perindopril erbumine.

The present invention relates to a new process for the preparation of pure perindopril erbumine. The present invention also relates to a new process for the preparation of crystalline form D of perindopril erbumine.

Perindopril and its pharmaceutically acceptable salts are known as angiontensin converting enzyme inhibitors and are used in the treatment of cardiovascular diseases, especially in the treatment of hypertension and heart failure. Perindopril is chemically known as (2S,3aS,7aS)-((2-(1-(ethoxycarbonyl)-(S)-butylamino)-(S)-propionyl)octahydro-indole-2-carboxylic acid and can be represented by formula (I).

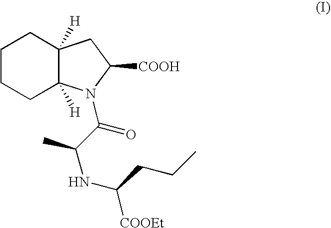

Perindopril was first disclosed in EP 0049658 B1 and U.S. Pat. No. 4,508,729 as optically pure S,S,S,S,S isomer and in the form of sodium salt. Numerous later patents and patent applications, such as EP 0308341 B1, EP 1279665 A1, EP 1333026 A1, WO 2004/099236 describe various processes for the preparation of perindopril.

tert-Butylamine salt of perindopril, known as perindopril erbumine, which is widely used in pharmaceutical products, was first disclosed in EP 0308341 B1. Perindopril erbumine may be obtained in different crystalline forms depending on crystallization conditions, e.g. solvent system, perindopril erbumine concentration and cooling kinetics. EP 1296947 B1 discloses crystallization of perindopril erbumine crystalline form a from ethyl acetate, EP 1294689 A discloses crystallization of perindopril erbumine crystalline form β from dichloromethane or ethyl acetate, EP 1296948 B1 discloses crystallization of perindopril erbumine crystalline form γ from chloroform, and WO 2004/113293 discloses crystallization of perindopril erbumine crystalline form δ and crystalline form ε. Crystalline form ε is obtained by crystallization from tert-butyl methyl ether containing 1.5 to 2.5% (vol/vol) of water whereas crystalline form δ is obtained from form E by azeotropic distillation.

EP 0308341 B1 describes an industrial process for the preparation of perindopril by coupling of protected (2S,3aS,7aS)-2-octahydroindole-2-carboxylic acid with N—[(S)-1-carbethoxybutyl]-(S)-alanine in the presence of dicyclohexylcarbodiimide and 1-hydroxybenzotriazole. Perindopril erbumine is obtained from crude perindopril by crystallization after adding of tert-butylamine. The drawback of this process is the formation of by-products derivable from dicyclohexylcarbodiimide, which are difficult to remove. For this reason, additional purification steps are needed to obtain pure perindopril and/or pure perindopril erbumine.

In addition to the by-products formed during the process of the preparation, also degradation products of perindopril are present as impurities in crude perindopril. Perindopril and its salts are chemically highly sensitive compounds and are susceptible to degradation via a) isomerisation at some chiral centres, b) hydrolysis of the side-chain ester group and/or c) intramolecular cyclization to form diketopiperazines. Two most critical diketopiperazines are (R)-ethyl 2-((3S,5aS,9aS,10aS)-3-methyl-1,4-dioxo-decahydropyrazino[1,2-a]indol-2(1H)-yl)pentanoate noted as diketopiperazine I and 2-((3S,5aS,9aS,10aR)-3-methyl-1,4-dioxo-decahydropyrazino[1,2-a]indol-2(1H)-yl)pentanoic acid noted as diketopiperazine II (formulas as depicted below), also indicated in European Pharmacopoea 5.1 as impurities F and C, respectively.

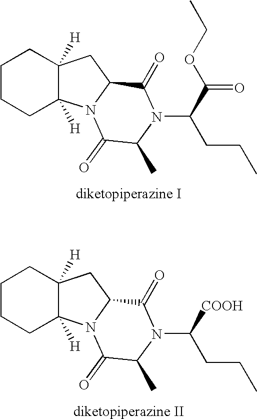

WO 01/58868 describes an improved process for the preparation of perindopril from (2S,3aS,7aS)-octahydroindole-2-carboxylic acid benzyl ester para-toluenesulfonate and N—[(S)-ethoxycarbonyl-1-butyl]-(S)-alanine in the presence of dicylohexylcarbodiimide, 1 hydroxybenzotriazole and optionally triethylamine. The formation of by-products derivable from dicyclohexylcarbodiimide is diminished by strict control of amounts of reagents used in the process. However, described process per se does not solve the problem of other impurities.

The problems of impurities derivable from dicyclohexylcarbodiimide is completely solved by processes for the preparation of perindopril that avoid use of dicyclohexylcarbodiimide. For example, such processes are described in patent applications EP 1279665 A1 and EP 1333026 A1. However, perindopril erbumine obtained by reaction of tert-butylamine with crude perindopril prepared according to the processes disclosed in these patent applications has to be additionally purified by different purification methods, such as thermal recrystallization or treatment with charcoal.

One can also mention WO 2004/046172 describing a process for the preparation of high pure perindopril erbumine, wherein a protected precursor of perindopril is deprotected in the presence of a base, e.g. tert-butylamine, to obtain perindopril salt, e.g. perindopril erbumine, directly without isolation of crude perindopril. A formation of diketopipirazine impurities during the manufacturing process is minimized due to short reaction time. However, other impurities have to be removed from obtained perindopril erbumine by additional crystallization step.

WO 2005/019173 discloses a process for the preparation of pure perindopril erbumine from crude perindopril by extracting aqueous solution of crude perindopril or its salt with suitable organic solvent at pH from 4.0 to 6.5, followed by separating of organic layer and preparing perindopril erbumine by adding tert-butylamine. The drawback of this process is a high number of steps that may results in low yield.

There is a continuing need for developing simple and effective process for the preparation of pure perindopril erbumine from crude perindopril, which does not require additional purification of obtained perindopril erbumine, and it is applicable at the industrial scale.

The present invention provides an improved process for the preparation of pure perindopril erbumine from crude perindopril, said improved process being especially effective in removing of diketopiperazine impurities. Furthermore, said process is simple and it is applicable at industrial scale.

A first object of the present invention is related to a process for the preparation of crystalline perindopril erbumine comprising the steps of:
(a) providing a solution of crude perindopril in wet aliphatic ester or in a mixture of wet aliphatic esters,
(b) adding tert-butylamine to the said solution,
(c) crystallizing perindopril erbumine, and
(d) isolating crystalline perindopril erbumine.

The process of the present invention allows to obtain a pure perindopril erbumine containing less than 0.20% (w/w) of diketopiperazine impurities, preferably containing less than 0.10% (w/w) of diketopiperazine impurities.

“Wet aliphatic ester” in the present invention means the aliphatic ester enriched or saturated with water. Preferably, wet aliphatic ester enriched with water contains from 1% (vol/vol) to 6% (vol/vol) of water, more preferably from 2% (vol/vol) to 4% (vol/vol) of water, most preferably from 2% (vol/vol) to 3% (vol/vol) of water.

In step (a), wet aliphatic ester is preferably selected from the group consisting of wet C₁-C₄ alkyl esters of C₁-C₄ aliphatic carboxylic acids. Preferably wet C₁-C₄ alkyl esters of C₁-C₄ aliphatic carboxylic acids include, but are not limited to, wet ethyl acetate, wet isopropyl acetate, wet butyl acetate and wet ethyl propionate. More preferably the wet aliphatic ester used in step (a) is wet ethyl acetate. Preferably, wet ethyl acetate used in step (a) contains from 1% (vol/vol) to 6% (vol/vol) of water, more preferably from 2% (vol/vol) to 4% (vol/vol) of water, most preferably from 2% (vol/vol) to 3% (vol/vol) of water.

The saturation of ethyl acetate can be executed by shaking it with water in a separation funnel with further separating of water phase. In order to avoid residual drops of water, ethyl acetate phase was cooled, preferably to temperature from −20° C. to −10° C., and carefully decanted from drops of water. Such ethyl acetate contains water in concentration slightly below saturation at room temperature and is one of the preferred wet aliphatic esters usable in step (a).

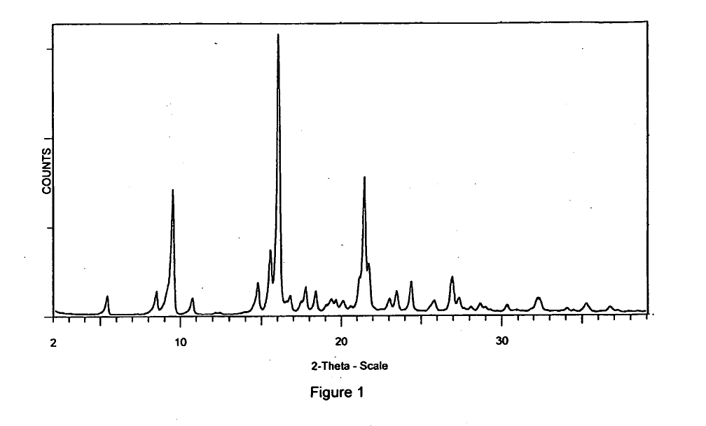

It has surprisingly been found that when the crystallization of perindopril erbumine is carried out in wet aliphatic ester or in a mixture of wet aliphatic esters according to the process of the present invention, the obtained perindopril erbumine crystalline form does not correspond to the crystalline forms known from the prior art, but to a new crystalline form of perindopril erbumine having a different X-ray powder diffraction pattern. New crystalline form of perindopril erbumine, named form D, has a powder x-ray diffraction pattern comprising the following characteristic reflection angles 2θ: 5.3±0.2°, 10.7±0.2°, 16.0±0.2°, 24.4±0.2° and 26.9±0.2°. New crystalline form D has a powder x-ray diffraction pattern as depicted in FIG. 1 having the following characteristic 2θ angles:

| Angle 2θ (°) | Relative intensity (%) |
|---|---|
| 5.3 | 4.7 |
| 8.4 | 7.0 |
| 9.4 | 34.4 |
| 10.7 | 5.0 |
| 14.7 | 15.7 |
| 15.5 | 33.3 |
| 16.0 | 100.0 |
| 16.7 | 6.6 |
| 17.7 | 9.2 |
| 18.3 | 10.2 |
| 21.1 | 22.1 |
| 21.5 | 59.3 |
| 21.7 | 25.6 |
| 23.0 | 6.0 |
| 23.5 | 9.0 |
| 24.4 | 12.7 |
| 25.7 | 6.9 |
| 26.9 | 18.1 |
| 27.3 | 6.7 |
| 28.1 | 2.8 |

Another embodiment object of the present invention is related to a process for the preparation of perindopril erbumine crystalline form D comprising the steps of:
(a) providing a solution of crude perindopril in wet aliphatic ester or in a mixture of wet aliphatic esters,
(b) adding tert-butylamine to the said solution,
(c) crystallizing perindopril erbumine crystalline form D, and
(d) isolating perindopril erbumine crystalline form D.

The process of the present invention allows to obtain a pure perindopril erbumine crystalline form D containing less than 0.20% (w/w) of diketopiperazine impurities, preferably containing less than 0.10% (w/w) of diketopiperazine impurities.

In step (a), said solution of crude perindopril in wet aliphatic ester or a mixture of wet aliphatic esters may be provided by dissolving crude perindopril in wet aliphatic ester or a mixture of wet aliphatic esters, optionally followed by removing of insoluble impurities by filtration. In another option, in step (a) also a solution, or a suspension of crude perindopril may be used for providing a solution of crude perindopril in wet aliphatic ester or in a mixture of wet aliphatic esters. Alternatively a solution of crude perindopril may be provided by an appropriate chemical reaction.

In a specific process according to the invention step (a) comprises the following sub-steps of:
(a1) dissolving crude perindopril in wet aliphatic ester or a mixture of wet aliphatic esters containing from 1% (vol/vol) to 6% (vol/vol) of water, and
(a2) removing of insoluble impurities by filtration.

In a specific process according to the invention step (a) comprises the following sub-steps of:
(a1′) dissolving crude perindopril in wet ethyl acetate containing from 2% (vol/vol) to 4% (vol/vol) of water, and
(a2′) removing of insoluble impurities by filtration.

In a specific process according to the invention step (a) comprises the following sub-steps of:
(a1′) dissolving crude perindopril in wet ethyl acetate saturated with water, and
(a2′) removing of insoluble impurities by filtration.

In step (b), tert-butylamine is added preferably at temperature between −20° C. and boiling point of tert-butylamine, more preferably at temperature from 20° C. to 40° C.

In a specific process according to the invention step (c) comprises the following sub-steps of:
(c1) heating the mixture obtained from step (b) up to the boiling point of the used aliphatic ester or the mixture of aliphatic esters,
(c2) filtration of the obtained boiling solution, and
(c3) cooling the obtained filtrate below 40° C. to obtain crystalline perindopril erbumine, preferably perindopril erbumine crystalline form D.

In sub-step (c3), the filtrate is preferably cooled below 20° C., more preferably to temperature from −10° C. to 0° C.

In a specific process according to the invention step (c) comprises the following sub-steps of:
(c1′) heating the mixture obtained from step (b) up to the boiling point of the used aliphatic ester or the mixture of aliphatic esters,
(c2′) filtration of the obtained boiling solution, and
(c3′) cooling the obtained filtrate below 20° C. to obtain crystalline perindopril erbumine, preferably perindopril erbumine crystalline form D.

In a specific process according to the invention, step (c) comprises the following sub-steps of:
(c1″) heating the mixture obtained from step (b) up to the boiling point of the used aliphatic ester or the mixture of aliphatic esters,
(c2″) filtration of the obtained boiling solution, and
(c3″) cooling the obtained filtrate to temperature from −10° C. to 0° C. to obtain crystalline perindopril erbumine, preferably perindopril erbumine crystalline form D.

Preferably, a mixture containing perindopril erbumine crystalline form D obtained after cooling according to sub-step (c3), (c3′) or (c3″) is left without agitation or stirring for about 15 to about 60 minutes, preferably for about 15 to about 45 minutes, more preferably for about 30 minutes, before the isolation of perindopril erbumine crystalline form D (step (d)) is carried out.

In a specific process according to the invention, step (d) comprises the following sub-steps:
(d1) isolation of crystalline perindopril erbumine, preferably perindopril erbumine crystalline form D, obtained from step (c) by filtration or centrifugation, preferably by filtration, and
(d2) drying of crystalline perindopril erbumine, preferably perindopril erbumine crystalline form D.

The filtration of crystalline perindopril erbumine, preferably perindopril erbumine crystalline form D, in sub-step (d1) is preferably performed at temperature below 0° C., more preferably at temperature from −20° C. to −10° C. in order to guarantee good yield and quality of obtained crystalline perindopril erbumine.

The preferred temperature of drying performed in sub-step (d2) is from 25° C. to 50° C., more preferred from 30° C. to 40° C. Preferably crystalline perindopril erbumine is dried to the constant weight.

In a specific process according to the invention step (d) comprises the following sub-steps:
(d1′) isolation of crystalline perindopril erbumine, preferably perindopril erbumine crystalline form D, obtained from step (c) by filtration at temperature from −20° C. to −10° C., and
(d2′) drying of crystalline perindopril erbumine, preferably perindopril erbumine crystalline form D, at temperature from 30° C. to 40° C.

A preferred process according to the invention comprises the following sub-steps of:
(a1) dissolving crude perindopril in wet aliphatic ester or in a mixture of wet aliphatic esters, containing from 1% (vol/vol) to 6% (vol/vol) of water,
(a2) removing of insoluble impurities by filtration,
(b) adding tert-butylamine to the solution obtained from step (a2), preferably at temperature between −20° C. and boiling point of tert-butylamine,
(c1) heating the mixture obtained from step (b) up to the boiling point of the used aliphatic ester or the mixture of wet aliphatic esters,
(c2) filtration of the obtained boiling solution,
(c3) cooling the obtained filtrate below 40° C. to obtain crystalline perindopril erbumine,
(d1) isolation of crystalline perindopril erbumine obtained from step (c3) by filtration or centrifugation, preferably by filtration, and (d2) drying of perindopril erbumine crystalline form D.

A preferred process for the preparation of perindopril erbumine crystalline form D according to the invention comprises the following sub-steps of:
(a1′) dissolving crude perindopril in wet ethyl acetate containing, and
(a2′) removing of insoluble impurities by filtration,
(b′) adding tert-butylamine to the solution obtained from step (a2) at temperature from 20° C. to 40° C.,
(c1″) heating the mixture obtained from step (b′) up to the boiling point of ethyl acetate,
(c2″) filtration of the obtained boiling solution,
(c3″) cooling the obtained filtrate to temperature from −10° C. to 0° C. to obtain perindopril erbumine crystalline form D,
(d1′) isolation of perindopril erbumine crystalline form D obtained from step (c3″) by filtration at temperature from −20° C. to −10° C., and
(d2′) drying of perindopril erbumine crystalline form D at temperature from 30° C. to 40° C.

A more preferred process for the preparation of perindopril erbumine crystalline form D according to the invention comprises the following sub-steps of:
(a1′) dissolving crude perindopril in wet ethyl acetate containing, and
(a2′) removing of insoluble impurities by filtration,
(b′) adding tert-butylamine to the solution obtained from step (a2) at temperature from 20° C. to 40° C.,
(c1″) heating the mixture obtained from step (b′) up to the boiling point of ethyl acetate,
(c2″) filtration of the obtained boiling solution,
(c3″) cooling the obtained filtrate to temperature from −10° C. to 0° C. to obtain perindopril erbumine crystalline form D,
(c4″) left a mixture obtained from step (c3″) without agitation for 15 to 60 minutes,
(d1′) isolation of perindopril erbumine crystalline form D obtained from step (c4″) by filtration at temperature from −20° C. to −10° C., and
(d2′) drying of perindopril erbumine crystalline form D at temperature from 30° C. to 40° C.

Optionally, perindopril erbumine crystalline form D obtained according to the process of the present invention may be further recrystallized from wet aliphatic ester, preferably from wet C₁-C₄ alkyl ester of C₁-C₄ aliphatic carboxylic acid or a mixture thereof.

The crystallization of perindopril erbumine from wet aliphatic ester according to the present invention, which preferably gives new crystalline form D, is an excellent method for removing most of diketopiperazine impurities as it is shown in Table 1.

TABLE 1
Amount of diketopiperazine impurities present in perindopril
erbumine prepared according to the Example 2

| | | Perindopril erbumine | Perindopril erbumine |
|---|---|---|---|
| | Crude | form D after 1st | form D after 2nd |
| | perindopril | crystallization | crystallization |
| Area % | 96.52% | 99.61% | 99.84% |
| Diketopiperazine I | 2.33% | 0.14% | 0.06% |
| Diketopiperazine II | 0.54% | 0.03% | 0.01% |

Another object of the present invention is related to use of perindopril erbumine crystalline form D, preferably containing less than 0.20% (w/w), more preferably less than 0.10% (w/w), of diketopiperazine impurities, for the preparation of pure perindopril erbumine crystalline form a or any other known crystalline form, preferably for the preparation of high pure perindopril erbumine crystalline form a or any other known crystalline form, wherein said pure or high pure perindopril erbumine crystalline form a or any other known crystalline form, preferably containing less than 0.20% (w/w), more preferably less than 0.10% (w/w), of diketopiperazine impurities.

Another object of the present invention is related to use of crystalline perindopril erbumine, preferably perindopril erbumine crystalline form D, prepared according to the process of the present invention for the preparation of perindopril erbumine crystalline form a or any other known crystalline form, preferably containing less than 0.20% (w/w), more preferably less than 0.10% (w/w), of diketopiperazine impurities.

Another object of the present invention is related to any of the processes as described above, wherein in a further step the perindopril erbumine crystalline form D, preferably containing less than 0.20% (w/w), more preferably less than 0.10% (w/w), of diketopiperazine impurities, as obtained after step (d) or sub-step (d1), (d1′), (d2) or (d21) is transformed to perindopril erbumine crystalline form a or any other known crystalline form, preferably containing less than 0.20% (w/w), more preferably less than 0.10% (w/w), of diketopiperazine impurities.

Another object of the present invention is related to a method of purifying perindopril erbumine comprising thermal recrystallization of perindopril erbumine from wet aliphatic ester or a mixture of wet aliphatic esters, preferably containing from 1% (vol/vol) to 6% (vol/vol) of water or from wet aliphatic ester saturated with water, more preferably containing from 2% (vol/vol) to 4% (vol/vol).

Wet aliphatic ester used for thermal recrystallization of perindopril erbumine is preferably selected from the group consisting of wet C₁-C₄ alkyl esters of C₁-C₄ aliphatic carboxylic acids. Preferably wet C₁-C₄ alkyl esters of C₁-C₄ aliphatic carboxylic acids include, but are not limited to, wet ethyl acetate, wet isopropyl acetate, wet butyl acetate and wet ethyl propionate. More preferably the wet aliphatic ester used for thermal recrystallization of perindopril erbumine is wet ethyl acetate.

A preferred method of purifying perindopril erbumine comprises the step of:
(ai) dissolving perindopril erbumine in wet aliphatic ester or in a mixture of wet aliphatic esters, preferably in wet aliphatic ester containing from 1% (vol/vol) to 6% (vol/vol) of water or in wet aliphatic ester or in a mixture of wet aliphatic esters saturated with water,
(bi) optionally removing of insoluble impurities by filtration,
(ci) heating the obtained mixture up to the boiling point of the used aliphatic ester or the mixture of aliphatic esters,
(di) optionally filtration of the obtained boiling solution,
(ei) cooling the obtained filtrate below 40° C., preferably to a temperature from −10° C. to 0° C.,
(fi) isolation of perindopril erbumine crystalline form D obtained from step (ei) by filtration or centrifugation, preferably by filtration at temperature from −20° C. to −10° C., and
(gi) drying of perindopril erbumine crystalline form D, preferably at temperature from 30° C. to 40° C.

Another object of the present invention is related to any of the processes as described above, wherein in a further step the crystalline perindopril erbumine, preferably, perindopril erbumine crystalline form D, as obtained after step (d), (d2), (d21) or (gi) is formulated into a pharmaceutically acceptable dosage form, in particular wherein said dosage form is a tablet, pill, capsule or injectable.

Another object of the present invention is crystalline perindopril erbumine obtained by the any process for the preparation of perindopril erbumine according to the present invention.

Another object of the present invention is crystalline perindopril erbumine obtainable by the any process for the preparation of perindopril erbumine according to the present invention.

Another object of the present invention is perindopril erbumine crystalline form D obtained by the any process for the preparation of perindopril erbumine crystalline form D according to the present invention.

Another object of the present invention is perindopril erbumine crystalline form D obtainable by the any process for the preparation of perindopril erbumine crystalline form D according to the present invention.

Another object of the present invention is related to pharmaceutical compositions comprising a therapeutically effective amount of perindopril erbumine containing less than 0.20% (w/w), preferably less than 0.10% (w/w), of diketopiperazine impurities together with one or more pharmaceutically acceptable carriers or other excipients.

A therapeutically effective amount of perindopril salt is the amount of perindopril salt, which comprises an amount of perindopril which is appropriate in a dosage form useful to treat hypertension or cardiovascular diseases. In general, a pharmaceutically effective amount of perindopril is 1 to 15 mg of perindopril, preferably 2 to 8 mg.

Pharmaceutically acceptable excipients may be selected from the group consisting of binders, diluents, disintegrating agents, stabilizing agents, preservatives, lubricants, fragrances, flavoring agents, sweeteners and other excipients known in the field of the pharmaceutical technology. Preferably, carriers and excipients may be selected from the group consisting of hydroxypropylcellulose, lactose, microcrystalline cellulose, calcium carbonate, starch, colloidal silicone dioxide, sodium starch glycolate, talc, magnesium stearate, polyvinylpyrrolidone, and other excipients known in the field of the pharmaceutical technology.

Optionally, the pharmaceutical compositions of the invention may be combination products comprising one or more additional pharmaceutically active components in addition to perindopril. Preferably, an additional pharmaceutically active component is a diuretic, e.g. indapamide.

Suitable pharmaceutical compositions are solid dosage forms, such as tablets with immediate release or sustained release of the active principle, effervescent tablets or dispersion tablets and capsules.

The pharmaceutical compositions may be prepared by methods known in the field of the pharmaceutical technology.

In another embodiment the present invention relates to use of perindopril erbumine containing less than 0.20% (w/w), preferably less than 0.10% (w/w), of diketopiperazine impurities for the preparation of a pharmaceutical composition for use in the treatment of cardiovascular diseases, e.g. hypertension or heart failure.

In another embodiment the present invention relates to a method for the treatment of cardiovascular diseases, e.g. hypertension or heart failure, comprising administering a therapeutically effective amount of perindopril erbumine containing less than 0.20% (w/w), preferably less than 0.10% (w/w), of diketopiperazine impurities.

The following examples illustrate the invention, but do not limit it in any way.

FIG. 1 represents X-ray diffraction diagram of perindopril erbumine crystalline form D obtained according to the process of the present invention.

### EXAMPLE 1

### Preparation of Crude Perindopril

A mixture of 9.54 g of (2S,3aS,7aS)-2-carboxyperhydroindole benzyl ester, 7.26 g of N—((S)-1-carbetoxybutyl)-(S)-alanine and 12.7 g of O-(benzotriazol-1-yl)-N,N,N′,N′-tetramethyluronium hexafluorophosphate in 225 ml of acetonitrile is stirred at room temperature for 30 min, then 560 ml of brine is added. The product is extracted twice with 400 ml of ethyl acetate, combined extracts are washed first with 800 ml of water, acidified with concentrated hydrochloric acid and then with 1.5 l of water. Organic phase is dried over anhydrous sodium sulphate and evaporated at 40° C. in vacuo to yield 13.5 g (88%) of benzyl (2S,3aS,7aS)-((2-(1-(ethoxycarbonyl)-(S)-butylamino)-(S)-propionyl)octahydroindole-2-carboxylate (benzyl ester of perindopril).

Crude benzyl ester of perindopril (13.5 g) is dissolved in 300 ml of methanol. To the solution is added 1.35 g of catalyst (10% palladium on charcoal). The mixture is stirred at room temperature under moderate flow of hydrogen for further 5 hours. The catalyst is then filtered off, washed with 50 ml of methanol and the solution is evaporated at 50° C. in vacuum. The obtained residue is crude perindopril as a clear colourless oily compound (2.33% of diketopiperazine 1, 0.54% of diketopiperazine II).

### EXAMPLE 2

### Preparation of Perindopril Erbumine from Perindopril

Perindopril obtained from example 1 (4.0 g, 2.33% of diketopiperazine I, 0.54% of diketopiperazine II) is dissolved in wet ethyl acetate (40 ml, water content 4% (vol/vol)). Insoluble impurities are filtered off, to the filtrate tert-butylamine (1.5 ml) is added under stirring at room temperature and the mixture is heated to reflux. The boiling solution is filtered and cooled to 6° C. The product is precipitated and after 30 minutes it is filtered and dried in vacuo at 40° C. for 24 hours to obtain perindopril erbumine in crystalline form D (2.5 g, 0.14% of diketopiperazine I, 0.03% of diketopiperazine II).

### EXAMPLE 3

### Preparation of Saturated Wet Ethyl Acetate

Ethyl acetate (100 ml) is shaken with 10 ml of water and water phase is removed. Ethyl acetate is cooled to −10° C. and pumped to the other vessel avoiding water sticked on the walls let to be warmed to room temperature.

Preparation of Perindopril Erbumine from Perindopril

Perindopril obtained from example 1 (4.0 g) is dissolved in ethyl acetate (40 ml), prepared as above. Insoluble impurities are filtered off, to the filtrate tert-butylamine (1.5 ml) is added under stirring at room temperature and the mixture is heated to reflux. The boiling solution is filtered and cooled to 0° C. The product is precipitated and after 30 minutes it is filtered and dried in vacuo at 40° C. for 24 hours to obtain perindopril erbumine in crystalline form D (2.9 g).

### EXAMPLE 4

### Preparation of Perindoprile Erbumine Form D from Perindopril Erbumine Form α

The mixture of perindopril erbumine (5 g) and wet ethyl acetate (30 ml), prepared as in Example 3, is heated to reflux under stirring. The solution is optionally filtered and cooled to 0° C. The product is precipitated. After 30 minutes the obtained suspension is filtered and the precipitate is dried in vacuo at 40° C. for 24 hours to yield perindopril erbumine crystalline form D (4.15 g).

### EXAMPLE 5

### Preparation of Perindoprile Erbumine Form D from Perindopril Erbumine Form α

The mixture of perindopril erbumine (5 g) and wet isopropyl acetate (prepared from 30 ml of isopropyl acetate and 1 ml of water) is heated to reflux under stirring. The solution is optionally filtered and cooled to −10° C., when upon it is left for 1 hour at −10° C. without agitation. The obtained suspension is filtered and the precipitate is dried in vacuo at 40° C. for 24 hours to yield perindopril erbumine crystalline form D.

Analytical data in examples are achieved by the following hardware:

Powder X-ray diffraction spectra of the sample is recorded on Siemens D-5000 with reflexion technique: CuKα radiation, range from 2° to 37° 2θ, step 0.04° 2θ, integration time 1 sec.

Chromatographic conditions for diketopiperazines determination:
1. Mobile phase:
   A: dissolve 0.92 g of sodium heptanesulphonate in 1000 ml of water, add 1 ml of triethylamine and adjust to pH 2.0 with a mixture of perchloric acid and water
   B: acetonitrile
2. Column: C8, 4 μm, pore size of 6 nm, 250×4.0 mm (Merck Supersphere 60 RP-8)
3. Conditions: temperature: 70° C., flow rate: 1.5 ml/min, wavelength: 215 nm, injection volume: 20 μl, gradient table:

| t | % A | % B |
|---|---|---|
| 0 | 73 | 27 |
| 8 | 73 | 27 |
| 25 | 40 | 60 |
| 30 | 40 | 60 |
| 40 | 20 | 80 |
| 45 | 0 | 100 |
| 50 | 73 | 27 |

4. Relative retention with reference to perindopril (about 11 min):
   diketopiperazine II-0.56
   diketopiperazine I-1.7
5. Equipment: Waters Alliance 2695 separations module, detector PDA 2996, software Empower 5.0

1. A process for the preparation of crystalline perindopril erbumine comprising the steps of:
(a) providing a solution of crude perindopril in wet aliphatic ester or in a mixture of wet aliphatic esters,
(b) adding tert-butylamine to the said solution,
(c) crystallizing perindopril erbumine, and
(d) isolating crystalline perindopril erbumine.

2. A process according to claim 1, wherein said wet aliphatic ester is selected from the group consisting of wet C₁-C₄ alkyl esters of C₁-C₄ aliphatic carboxylic acids.

3. A process according to claim 1, wherein said wet aliphatic ester is wet ethyl acetate.

4. A process according to claim 3, wherein said wet ethyl acetate contains from 1% (vol/vol) to 6% (vol/vol) of water.

5. A process according to claim 3, wherein said wet ethyl acetate contains from 2% (vol/vol) to 4% (vol/vol) of water.

6. A process according to claim 3, wherein said wet ethyl acetate is prepared by saturation with water at temperature from −20° C. to −10° C.

7. A process according to claim 1, wherein in step (b) tert-butylamine is added at temperature from 20° C. to 40° C.

8. A process according to claim 1, wherein step (c) comprises the sub-steps of:
(c1) heating the mixture obtained from step (b) up to the boiling point of the used aliphatic ester or the mixture of aliphatic esters,
(c2) filtration of the obtained boiling solution, and
(c3) cooling the obtained filtrate below 40° C. to obtain crystalline perindopril erbumine.

9. A process according to claim 8, wherein in sub-step (c3) said filtrate in cooled to the temperature from −10° C. to 0° C.

10. A process according to claim 1, wherein step (d) comprises the sub-steps of:
(d1) isolation of crystalline perindopril erbumine obtained from step (c) by filtration or centrifugation, and
(d2) drying of crystalline perindopril erbumine.

11. A process according to claim 10, wherein said filtration in sub-step (d1) is performed at temperature below 0° C.

12. A process according to claim 10, wherein said filtration in sub-step (d1) is performed at temperature from −20° C. to −10° C.

13. A process according to claim 1, wherein said crystalline perindopril erbumine obtained from step (d) contains less than about 0.20% (w/w) of diketopiperazine impurities.

14. A process according to claim 1, wherein said crystalline perindopril erbumine is perindopril erbumine crystalline form D.

15. A process according to claim 14, wherein said perindopril erbumine crystalline form D has a powder x-ray diffraction pattern comprising the following characteristic reflection angles 2θ: 5.3±0.2°, 10.7±0.2°, 16.0±0.2°, 24.4±0.2° and 26.9±0.2°.

16. A process according to claim 14, wherein said perindopril erbumine crystalline form D has a powder x-ray diffraction pattern comprising the following characteristic 2θ angles:

| Angle 2θ (°) | Relative intensity (%) |
|---|---|
| 5.3 | 4.7 |
| 8.4 | 7.0 |
| 9.4 | 34.4 |
| 10.7 | 5.0 |
| 14.7 | 15.7 |
| 15.5 | 33.3 |
| 16.0 | 100.0 |
| 16.7 | 6.6 |
| 17.7 | 9.2 |
| 18.3 | 10.2 |
| 21.1 | 22.1 |
| 21.5 | 59.3 |
| 21.7 | 25.6 |
| 23.0 | 6.0 |
| 23.5 | 9.0 |
| 24.4 | 12.7 |
| 25.7 | 6.9 |
| 26.9 | 18.1 |
| 27.3 | 6.7 |
| 28.1 | 2.8 |

17. A process for the preparation of perindopril erbumine crystalline form D comprising the steps of:
(a1′) dissolving crude perindopril in wet ethyl acetate saturated with water, and
(a2′) removing of insoluble impurities by filtration,
(b′) adding tert-butylamine to the solution obtained from step (a2) at temperature from 20° C. to 40° C.,
(c1″) heating the mixture obtained from step (b′) up to the boiling point of ethyl acetate,
(c2″) filtration of the obtained boiling solution,
(c3″) cooling the obtained filtrate to temperature from −10° C. to 0° C. to obtain perindopril erbumine crystalline form D,
(d1′) isolation of perindopril erbumine crystalline form D obtained from step (c3″) by filtration at temperature from −20° C. to −10° C., and
(d2′) drying of perindopril erbumine crystalline form D at temperature from 30° C. to 40° C.

18. Use of crystalline perindopril erbumine, prepared according to claim 1 for the preparation of perindopril erbumine crystalline form α or any other known crystalline form.

19. A method of purifying perindopril erbumine comprising thermal recrystallization of perindopril erbumine from wet aliphatic ester or a mixture of wet aliphatic esters.

20. Crystalline perindopril erbumine obtained by the process according to claim 1.

21. Perindopril erbumine crystalline form D obtained by the process according to claim 14.

22. Perindopril erbumine crystalline form D obtainable by the process according to claim 17.

23. A process according to claim 1, wherein in a further step the crystalline perindopril erbumine as obtained after step (d), is formulated into a pharmaceutically acceptable dosage form.

24. A process according to claim 17, wherein in a further step the perindopril erbumine crystalline form D as obtained after step (d2′), is formulated into a pharmaceutically acceptable dosage form.
